(19) United States
(12) Patent Application Publication
(10) Pub. No.: US 2025/0221942 A1
(43) Pub. Date: Jul. 10, 2025

(54) METHOD OF ISOLATING A HIGH NUMBER OF EXOSOME EXTRACTS WITH HIGH-ACTIVITY FROM ANIMAL-DERIVED CORD BLOOD OR FETAL ANIMAL OR ANIMAL MILK OR OTHER ANIMAL-DERIVED BODY FLUIDS TO ALLOW HIGH-ACTIVITY EXOSOME-LIPOSOME HYBRIDIZATION

(71) Applicant: Kemal Tunc TIRYAKI, Istanbul (TR)

(72) Inventor: Kemal Tunc TIRYAKI, Istanbul (TR)

(21) Appl. No.: 18/699,598

(22) PCT Filed: Dec. 24, 2021
(86) PCT No.: PCT/TR2021/051494

§ 371 Date: Apr. 9, 2024

(51) Int. Cl. A61K 9/50; A61K 8/14

(52) U.S. Cl. CPC A61K 9/5068; A61K 8/14

(57) ABSTRACT

A method of isolating a high number of exosome extracts with high-activity from animal-derived cord blood or fetal animal or animal milk or other animal-derived body fluids to allow high-activity exosome-liposome hybridization is provided. The method is performed as follows: the animal cord blood is transferred into a tube/blood bag containing 1-3% Anticoagulant and shaken in reverse at a 90-degree angle gently, then the blood sample is transferred into a large cylindrical chamber, and is pushed repeatedly through an air-tight chamber with medical grade glass beads to achieve emulsification, at least 1 time per second for at least 20 repetitions. The plasma of the blood obtained in the above step is centrifuged at 2,000-3,000 G/min for 2-6 minutes to obtain 2 phases, the plasma fragment containing large and small proteins and exosomes is separated, and the exosomes in the plasma fragment are isolated by using a polymer-based precipitation method.

### CROSS REFERENCE TO THE RELATED APPLICATIONS

This application is the national phase entry of International Application No. PCT/TR2021/051494, filed on Dec. 24, 2021, the entire contents of which are incorporated herein by reference.

### TECHNICAL FIELD

The invention relates to a method where/in which high number and high-activity exosomes are isolated by activating animal-derived cord blood or fetal animal blood or animal milk or other animal-derived body fluids with the physical, chemical or biological methods and then the isolated and activated exosomes are hybridized with cationic liposomes to obtain a hybrid molecule with high activity. The resulting hybrid molecule can be used in pharmaceutics and cosmetics.

### BACKGROUND

For many years, it was believed that aging develops autonomously over time. However, the studies indicate that reducing the old cell mass may slow down the aging process and even reverse it over time, thereby providing the cellular rejuvenation.

Heterochronic parabiosis, i.e., the studies in which the old and young individuals are joined with an artery gene bond and the plasma is transferred, is the most significant proof showing that the process is not autonomous. The parabiosis studies showed that replacing the blood of the old individuals with the blood of the young individuals on a regular basis can prevent the changes caused by the degenerative, i.e., destructive effects in the elderly, and that it is even possible to rejuvenate over time, but when the system is reversed, i.e., the blood of old individuals is used in the young individuals, this may induce aging. These studies show that the aging is non-autonomous and the old blood cells accumulate and send the aging signal to the young blood cells.

The studies revealed that all these factors are due to the extracellular vesicles, i.e., exosomes which are secreted by the old or young stem cells and transferred to the blood.

The extracellular vesicles are roughly divided into two groups, namely the exosomes of 30-120 nm and nanoparticles larger than 120 nm. The vesicles contain mRNA, miRNA, growth factors, and immune regulators, and the membranes thereof contain immunoactive (such as MHC) molecules. The transport of the extracelularvesicles is carried out through the blood in the body. In this transport, the blood is the primary vehicle for the cell-cell communication.

The blood is a living liquid consisting of the blood cells, which circulates consistently in the vessels and is essential for the life. The blood cells consist of various shapes and plasma. The plasma in the exterior part constitutes 55% of the volume of the blood. 90% of the plasma consists of water and the remaining part consists of the organic and inorganic substances such as plasma proteins, amino acids, nanovesicles, carbohydrates, fats, hormones, urea, uric acid, lactic acid, enzymes, antibodies, and elements such as sodium, potassium, iodine, iron, and bicarbonate.

It is known that there are 300 different proteins in blood plasma. Some soluble proteins normally found in the intracellular fluids can migrate to the extracellular and intravascular fluids when the cell is damaged and communicate mostly with the nano-carriers called exosomes.

Exosomes are the small extracellular transport vesicles with 30-120 nanometer in diameter size, which are secreted into the environment by all known cells, play an active role in the communication of cells with each other, and carry the intercellular genetic material. These small vesicles can be taken into the cell by other cells, transfer DNA, RNA, or proteins from cell to cell and cause the genetic changes. Exosomes secreted from cells are considered to be one of the most important way for the cells to communicate with each other. The small nano-sized particles which are filled with the genetic material and have the potential to reverse the disease processes have entered into the literature as the first step in the individual-specific treatments.

Exosomes which can be obtained from all body fluids play an important role in many biological activities such as intercellular communication, signal transmission, genetic material transfer, and regulation of immunological response. Exosomes have the ability to repair the skin, rejuvenate the skin and reduce the inflammation, and also these nanoparticles can easily pass through the skin barrier.

Because of their immunosuppressive and immunomodulatory characteristics, the exosomes originating from the different cells have the therapeutic applications to allow the immunological function to be regulated in the case of autoimmune suppression and immunosuppression. In addition, since exosomes are the natural nano-carriers, they enable the design of new generation vaccines in which both the adjuvant and the antigen can be transported.

The intercellular communication is of vital importance and is carried out in different ways in organisms. The extracellular vesicles take part in the long-distance communication and carry the cell-specific substances such as DNA, RNA, and proteins. The extracellular vesicles are divided into 3 groups, apoptosomes, microvesicles and exosomes, according to their size and formation. For the characterization of the exosomes, some surface markers are tested using the flow cytometry method, some of which are CD9, CD63 and HSP70. Besides, these nanoparticles can be characterized by the immunoblotting method and size measurement. The immunizing roles of the dendritic cell-derived exosomes also demonstrate the therapeutic use of the exosomes. The differential centrifugation, immunoaffinity, ultrafiltration, and polymer-based precipitation are among the most widely used methods for obtaining the exosomes from animal and plant sources. Particular attention is paid to their role in the transportation of the bioactive compounds with low solubility and low bioavailability. Thus, the literature data suggest that the transportation of the bioactive compounds by the exosomes may be an alternative solution to the nanoparticles which have the disadvantages such as immune reactions, toxicity, need for modification and artificial synthesis.

On the other hand, the exosomes are the valuable biomaterials for the development of new nano-carriers as the functionally advanced drug delivery systems. There are the studies on creating the hybrid exosomes using the freezing-thawing method to control and modify the performance of the exosomal nano-carriers. It has been demonstrated that by hybridizing the exosomes embedded with a specific membrane protein isolated from the genetically modified cells with various liposomes, the new hybrid nano-carriers can change the interactions between cells, lipid composition, or properties of lipids.

The studies have focused on the exosome engineering with the aim of increasing the efficiency of the exosomes by the methods such as surface modification and hybridization with the synthetic nano-carriers such as liposomes. The hybrid exosomes, i.e., the entirely new biological nano-carriers, have the potential to be a new route to be used to transport the hydrophilic loads as well as hydrophobic lipids to the recipient cell. The hybrid exosomes are based on a technology that fuses the exosomes and liposomes through the membrane fusion and functions as a hybrid nanoparticle system.

With the hybridization technology, the exosomes can be combined with the liposomes loaded with the precursors required for the field in which they will be used (for example, for hair products, especially those filled with biotin). The advantages of the hybrid molecule formed by hybridizing the exosome and liposome cells are as follows.
It allows the active product to be out of the exosome extent and to fall within the cosmetic class.
It facilitates the entry of the active product into the cell.
It prevents the imitation of the active product.
The different active products can be produced for different purpose (hair, skin, wound, cellulite).
It can pass through the digestive system.

Naturally, the hybrid exosomes with these membrane modifications allow both the genetic material required for rejuvenation and the specific active substance required for that area to reach the target are more easily.

In the literature review related to the current technique, the application no. JP2014185090A is found regarding the hybridization of the exosome cells with the liposome cells. The application of the present art describes a method that allows the insertion of the bioactive substance into the exosome, in other words, its hybridization, in order to obtain an exosome-liposome hybrid molecule. The basic novelty on which the invention is based refers to mixing the physiologically active substance and the liposome encapsulating the exosome and subjecting it to a freezing/thawing step.

As a result, today, the exosome-liposome hybridization is a known method. However, the developments continue, which relate to allowing the hybrid molecule resulting from the hybridization of the exosome-liposome cells to be a high-activity molecule with the higher properties.

### SUMMARY

The object of the invention is to isolate the high number and high-activity exosomes by means of the physical, chemical or biological methods, compared to the hybrid exosomes obtained by the currently used methods for the isolation and hybridization of the exosomes, and also to obtain a hybrid molecule with high activity with these isolated exosomes. However, this result is not achieved by the novelty in the hybridization process or a new hybridization process. It is achieved by the method where/in which the activated exosome molecules are obtained. Thanks to the method of the invention, the exosome molecules which are isolated by increasing their activity and quantity by the biological, chemical or physical methods can be hybridized with the liposome molecule by the known methods and then the hybrid molecules with high activity can be obtained.

The present invention relates to a method where/in which the exosomes in the blood are isolated by increasing their activity and number by the physical, chemical or biological activation of the animal cord blood. Due to the high activity (with the increasing in activity and number) of the exosome cells to be obtained by the method of the invention, it is the object to obtain a more active hybrid molecule after the hybridization.

Here, the basic object is to increase the number and activity of the fetal exosomes in the cord blood by the physical, chemical or biological methods and to obtain a high number of the exosomes capable of carrying more biological materials. In this way, it is the object to obtain more active exosomes with higher number compared to the non-activated exosome-liposome hybrid molecules. The same object applies to the fetal animal blood or animal milk or other animal-derived body fluids.

One of the basic objects of the invention is to obtain high number of exosomes with high activity and to allow the new hybrid nano-carrier that will be formed after the hybridization with the liposome to have higher activity than the molecules hybridized by the normal processes.

Another object of the invention is to obtain more exosome cells from the same amount of animal-derived cord blood. The same object applies to the fetal animal blood or animal milk or other animal-derived body fluids.

The structural and characteristic features and all advantages of the invention will be understood more clearly thanks to the detailed description below. Therefore, the evaluation should be made taking into account said detailed description.

### BRIEF DESCRIPTION OF THE DRAWINGS

FIGS. 1A-1B Flow cytometry graphs in which the presence of the hybrid molecule obtained by the standard methods is determined by the surface markers CD 9 and CD63, respectively.

FIGS. 1C-1D Flow cytometry graphs in which the presence of the hybrid molecule obtained by the activation method described in the invention is determined by the surface markers CD 9 and CD63, respectively.

FIG. 2A Cell viability analysis of the Human Dermal Fibroblast (HDF) cells at 24, 48 and 72 hours after the treatment with the hybrid molecule which is obtained by the standard methods and applied at the different concentrations (100 and 200 μg/mL).

FIG. 2B Cell viability analysis of the Human Dermal Fibroblast (HDF) cells at 24, 48 and 72 hours after the treatment with the hybrid molecule which is obtained by the activation method described in the invention and applied at the different concentrations (100 and 200 μg/mL).

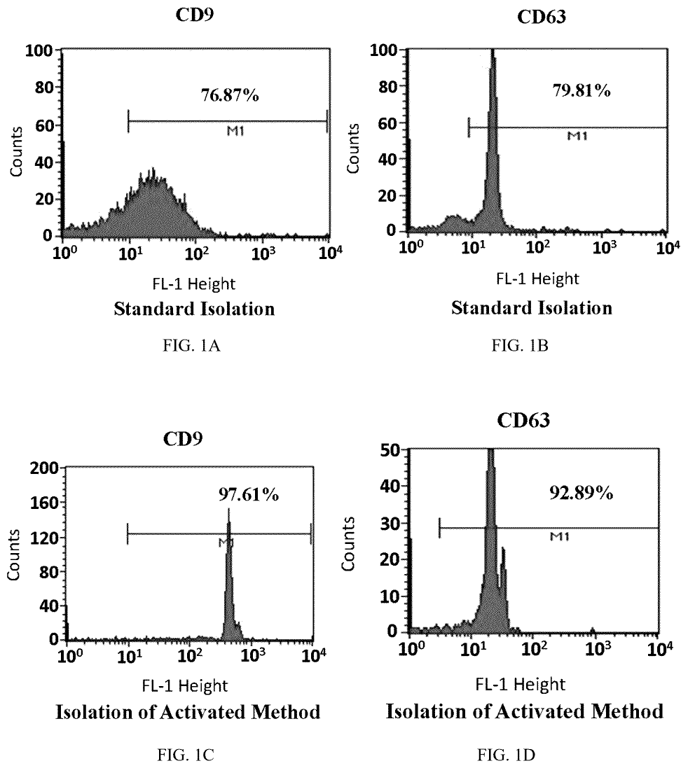

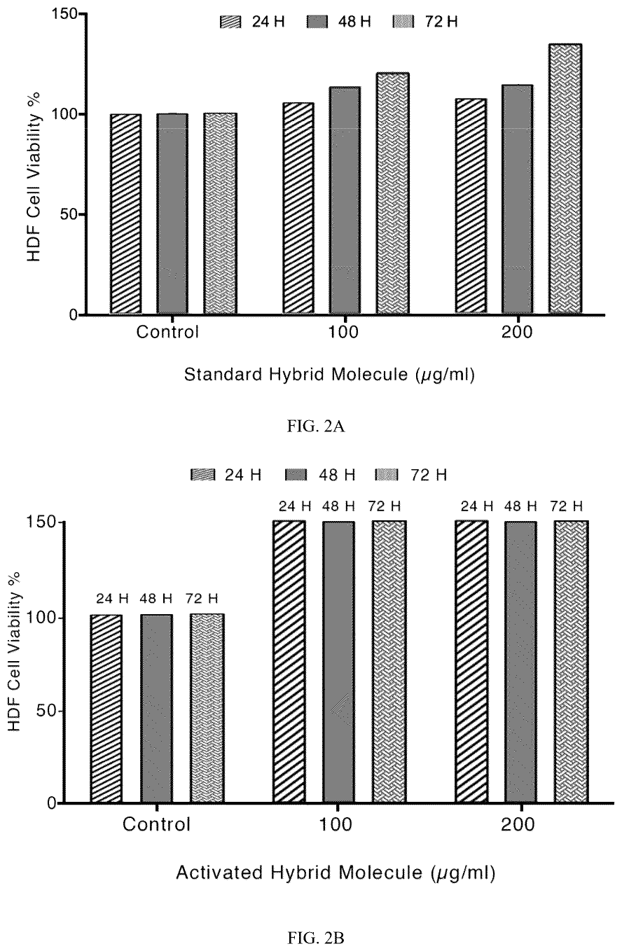

### DETAILED DESCRIPTION OF THE EMBODIMENTS

In this comprehensive study, a method of the invention will be explained, where/in which high number and high-activity exosome extracts are isolated from the animal-derived cord blood or fetal animal blood or animal milk or other animal-derived body fluids and then the obtained active exosomes are hybridized with liposomes to obtain a new hybrid molecule with increased activity. The method of the invention with the preferred embodiment thereof will only explained for a better understanding of the subject and without any limiting effect.

The activation method used in the method of the invention for obtaining the exosomes with increased activity can be physical, chemical, or biological. In order to understand how the invention is applied, the physical activation method is carried out as described below:
For the isolation (extraction) of the activated exosomes from the animal-derived cord blood, the cord blood circulating in the cords of the female animals giving birth is transferred to a tube/bag containing 1-3% Anticoagulant.
The transferred blood is gently shaken by turning it upside-down at an angle of 90°.
After completing the shaking, the blood sample is transferred into a large cylindrical chamber with a Luer Lock tip.
The cylindrical chamber filled with the blood is connected to another air-tight chamber having 2 Luer Lock inlets and containing the medical glass beads (grade glass beads).
An empty cylindrical chamber with a Luer Lock inlet is connected to the other end of the chamber containing the glass beads.
The blood is emulsified by passing it from one side to another through the glass beads, such as from the filled cylindrical chamber at one end of the chamber containing the glass beads to the empty cylindrical chamber at the other end thereof, and then vice versa, at least 1 time per second and with at least 20 repetitions. The passive exosomes in the blood cells which are physically emulsified by passing through the glass beads are also actively released into the external environment.
It is centrifuged at a speed of 2000-3000×g/min for 2-6 min to divide the blood plasma activated by passing through the glass beads into two phases. The red part in the lower part consists of the blood cells and cell debris, while the plasma fragment is formed in the upper part. The plasma fragment is a fluid which is rich in growth factors and consists of large and small proteins and exosomes. For the exosome isolation process, the plasma fragment is separated and collected in another chamber.
As is known in the literature, there are many exosome isolation methods. Some of these are the known methods such as immunoprecipitation, nanofiltration and ultracentrifugation. Within the scope of the invention, the exosome isolation by a polymer-based precipitation method was preferred. The polymer-based exosome isolation method has a distinct advantage over other exosome isolation methods since it can isolate the extracellular vesicles with high efficiency and purity in a shorter time and without requiring the special equipment.
In order to obtain the hybrid nanoparticles with high activity, a great number of the exosome molecules with increased activity obtained as described above are hybridized with the liposome molecules using the sonication method.

The novelty of the invention is in obtaining a higher number of exosomes with higher activity, compared to the normal isolation methods, a result of the physical, chemical or biological activation of the animal-derived cord blood before the hybridization process. The reason for the increase in the number of the exosomes is that the blood cells get stressed and release also the exosomes contained within the cell. As in the present situation, in the isolation processes performed without activating the blood cells, the exosomes contained within the cell are excreted without being isolated. If the cells are activated physically, chemically, or biologically before the isolation process, the cells also release the exosomes therein out of the cell. Thus, the exosomes which are both more active and higher in number are isolated. As a result of the hybridization of the resulting exosomes having high activity with the cationic liposomes, a hybrid molecule with high activity is obtained.

The steps described above based on the animal-derived cord blood can also be applied to the fetal animal blood or animal milk or other animal-derived body fluids.

The process is the same for the fetal animal blood. The only difference in the process of the exosome isolation from the animal milk or other animal-derived body fluids is that there is no need to transfer to the tube containing the anticoagulant and shake at a 90-degree angle. Furthermore, since there is no need to separate the plasma, the activated animal-derived body fluid or animal milk is subjected to the exosome isolation by the polymer-based precipitation method without being subjected to the centrifugation. In the next step, it is hybridized with the liposome molecule using the sonication method in the same way.

What is claimed is:

1. A method of isolating a high number of exosome molecules with a high activity from an animal-derived cord blood or a fetal animal blood or an animal milk or other animal-derived body fluids for a use in a hybridization process, comprising steps of:
obtaining the high number of the exosome molecules with the high activity as a result of a physical activation, a chemical activation, or a biological activation of the animal-derived cord blood or the fetal animal blood or the animal milk or the other animal-derived body fluids, and
forming a hybrid nanomolecule with a high activity by hybridizing the exosome molecules having the high activity with cationic liposomes.

2. The method of isolating the high number of the exosome molecules with the high activity according to claim 1, wherein the exosome molecules are isolated from the animal-derived cord blood and obtained by steps of:
transferring the animal-derived cord blood circulating in cords of female animals giving birth to a tube/bag containing 1-3% Anticoagulant for an isolation or an extraction of activated exosomes from the animal-derived cord blood,
gently shaking a transferred blood to be turned upside-down at a 90-degree angle,
after completing a shaking process, transferring a blood sample into a cylindrical chamber with a Luer Lock tip,
connecting the cylindrical chamber filled with the blood sample to a first end of an air-tight chamber comprising two Luer Lock inlets and medical grade glass beads by the Luer Lock tip,
connecting an empty cylindrical chamber to a second end of the air-tight chamber comprising the two Luer Lock inlets,
emulsifying blood molecules by pushing the blood sample from the cylindrical chamber filled with the blood sample at the first end of the air-tight chamber containing the medical grade glass beads to the empty cylindrical chamber at the second end thereof, and then vice versa, at least 1 time per second for at least 20 repetitions,
centrifuging a plasma of the blood sample activated by passing through the medical grade glass beads for 2-6 min at a speed of 2000-3000 G/min for obtaining two phases,
separating a plasma fragment consisting of large and small proteins and exosomes after a centrifugation process and isolating the exosomes in the plasma fragment by using a polymer-based precipitation method.

3. The method of isolating the high number of the exosome molecules with the high activity according to claim 1, wherein the exosome molecules are isolated from the fetal animal blood and obtained by steps of:
transferring a 0-6-month-old fetal animal blood into a tube containing 1-3% Anticoagulant,
gently shaking a transferred blood to be turned upside-down at a 90-degree angle,
after completing a shaking process, transferring a blood sample into a cylindrical chamber with a Luer Lock tip,
connecting the cylindrical chamber filled with the blood sample to a first end of an air-tight chamber comprising two Luer Lock inlets and containing the medical grade glass beads by the Luer Lock tip,
connecting an empty cylindrical chamber to a second end of the air-tight chamber comprising the two Luer Lock inlets,
emulsifying blood molecules by pushing the blood sample from the cylindrical chamber filled with the blood sample at the first end of the air-tight chamber containing the medical grade glass beads to the empty cylindrical chamber at the second end thereof, and then vice versa, at least 1 time per second for at least 20 repetitions,
centrifuging a plasma of the blood sample activated by passing through the medical grade glass beads for 2-6 min at a speed of 2000-3000 G/min for obtaining two phases,
separating a plasma fragment consisting of large and small proteins and exosomes as a result of a centrifugation process and isolating the exosomes in the plasma fragment by using a polymer-based precipitation method.

4. The method of isolating the high number of the exosome molecules with the high activity according to claim 1, wherein the exosome molecules are isolated from the animal milk and obtained by steps of:
transferring an animal milk sample into a cylindrical chamber with a Luer Lock tip,
connecting the cylindrical chamber filled with the animal milk sample to a first end of an air-tight chamber comprising two Luer Lock inlets and containing the medical grade glass beads by the Luer Lock tip,
connecting an empty cylindrical chamber to a second end of the air-tight chamber comprising the two Luer Lock inlets,
emulsifying molecules by pushing the animal milk sample from the cylindrical chamber filled with the animal milk sample at the first end of the air-tight chamber containing the medical grade glass beads to the empty cylindrical chamber at the second end thereof, and then vice versa, at least 1 time per second for at least 20 repetitions,
isolating exosomes of the animal milk sample activated by passing through the medical grade glass beads using a polymer-based precipitation method.

5. The method of isolating the high number of the exosome molecules with the high activity according to claim 1, wherein the exosome molecules are isolated from the other animal-derived body fluids and obtained by steps of:
transferring an animal-derived body fluid sample other than blood into a cylindrical chamber with a Luer Lock tip,
connecting the cylindrical chamber filled with the animal-derived body fluid sample other than blood to a first end of an air-tight chamber comprising two Luer Lock inlets and medical grade glass beads by the Luer Lock tip,
connecting an empty cylindrical chamber to a second end of the air-tight chamber comprising the two Luer Lock inlets,
emulsifying molecules by pushing the animal-derived body fluid sample other than blood from the cylindrical chamber filled with the animal-derived body fluid sample other than blood at the first end of the air-tight chamber containing the medical grade glass beads to the empty cylindrical chamber at the second end thereof, and then vice versa, at least 1 time per second for at least 20 repetitions,
isolating exosomes of the animal-derived body fluid sample other than blood activated by passing through the medical grade glass beads using a polymer-based precipitation method.
